(19) United States
(12) Patent Application Publication
(10) Pub. No.: US 2014/0249369 A1
(43) Pub. Date: Sep. 4, 2014

(54) IMAGING APPARATUS AND RIGID ENDOSCOPE

(71) Applicant: Shinichi Hanabusa, Tokyo (JP)

(72) Inventor: Shinichi Hanabusa, Tokyo (JP)

(73) Assignee: SERENDIPITY CO., LTD, Itabashi-ku, Tokyo (JP)

(21) Appl. No.: 14/349,285

(22) PCT Filed: Mar. 26, 2012
(86) PCT No.: PCT/JP2012/002086

§ 371 Date: Apr. 2, 2014

(30) Foreign Application Priority Data
Oct. 3, 2011 (JP) 2011-219663

(51) Int. Cl. A61B 1/045; A61B 1/06

(52) U.S. Cl. CPC A61B 1/045; A61B 1/06
USPC 600/109

(57) ABSTRACT

An aim is to provide an imaging apparatus that can reduce the size without being constrained by the size of a mechanism that drives the apparatus, and can accurately move a field of view over a wide range in a narrow space. The apparatus includes an image-wise light receiving means, a spherical housing that holds therein the image-wise light receiving means, a base that supports the spherical housing and enables the spherical housing to freely move along a surface thereof, a drive wire having an end fixed to the spherical housing, and a drive section to which the other end of the drive wire is fixed to drive the free movement of the spherical housing via the drive wire.

### BACKGROUND

1. Field of the Disclosure

The present disclosure relates to an imaging apparatus and, more particularly, to an imaging apparatus that is small in size and able to expand the range of rotational movement in a field of view.

2. Discussion of the Background Art

A conventional imaging apparatus used in a narrow space in performing an endoscopic examination, piping inspection, or the like is engineered to reduce the size of the imaging section and to ensure any direction in a field of view. For example, a CCD camera and a lens are housed in a spherical actuator, and the rotor that forms the spherical actuator is held by a plurality of stators. The stators are permitted to generate torque around their respective axes to realize the movement of the CCD camera based on three degrees of freedom (e.g., see Patent Document 1).

For example, a known imaging apparatus has a spherical housing that houses an imaging means therein, while the housing is mounted with a plurality of stacked piezoelectric actuators, to thereby rotatably move the whole spherical housing to any direction (e.g., see Patent Document 2). For example, a known endoscope includes a power-operating means that power-operates a bendable portion and a movable portion, such as a treatment-tool elevating device, using an operating wire inserted from an operating section (e.g., see Patent Document 3).

Patent Documents

Patent Document 1 JP-A-H09-238485

Patent Document 2 JP-A-H05-344951

Patent Document 3 JP-A-H08-280606

However, in Patent Documents 1 and 2, there is a problem that size reduction of the apparatus as a whole is constrained by the size of the drive mechanism, such as the stators, because the stators or the stacked piezoelectric actuator are directly mounted to the spherical actuator or the spherical housing inserted into a narrow space. Further, when stators, or ultrasonic motors, in particular, are used, it is difficult to achieve driving with a highly accurate angular velocity and thus the work using the imaging apparatus may be hindered.

Further, in Patent Document 3, since an insertion tube having flexibility is flexed in moving a field of view, a space is necessary for the insertion tube to be flexed in a narrow observation area. If a sufficient field of view is to be ensured, imaging targets are problematically limited to those which have a space where the insertion tube can be flexed.

In light of the foregoing problems, the present disclosure has an object of providing an imaging apparatus that can reduce the size without being constrained by the size of a mechanism for driving the apparatus and is able to accurately move a field of view over a wide range in a narrow space.

### SUMMARY

In order to achieve the above object, an imaging apparatus related to the present disclosure has a most principal feature that one end of a drive wire is fixed to a surface of a spherical housing that holds an image-wise light receiving means such as of an image sensing device, the other end of the drive wire is fixed to a drive section, and the drive section is allowed to freely move the spherical housing via the drive wire.

Specifically, the drive section is provided at a position far from the spherical housing through the drive wire to enable free movement of the spherical housing without the necessity of directly mounting a drive section having an imaging means to the spherical housing. The spherical housing only has to be supported by a base that enables the spherical housing to freely move along the surface thereof.

In the imaging apparatus related to the present disclosure, the spherical housing is freely moved via the drive wire. Accordingly, a drive section is no longer required to be provided to the spherical housing itself or in the vicinity of the spherical housing. Thus, the size of an imaging section that is inserted into a narrow observation area can be advantageously reduced without being constrained by the size of a mechanism that drives the imaging section.

### BRIEF DESCRIPTION OF THE DRAWINGS

FIG. 1 shows by (a) a schematic diagram illustrating an imaging apparatus related to the present disclosure and by (b) a schematic diagram of the imaging apparatus as viewed from a direction A indicated in (a);

FIG. 2 is a partially enlarged perspective view illustrating a tip portion of the imaging apparatus related to the present disclosure;

FIG. 3 is a partially enlarged side cross-sectional view of the tip portion of the imaging apparatus related to the present disclosure;

FIG. 4 is a top cross-sectional view illustrating a driving section;

FIG. 5 is a schematic diagram illustrating operation of a spherical housing of the imaging apparatus related to the present disclosure, specifically showing by (a) that a narrow angle of 30° is formed between two sides that connect the center of the spherical housing to fixing parts of mutually adjacent wires, the parts being formed on the surface of the spherical housing, and by (b) that the narrow angle is 240°;

FIG. 6 is a side cross-sectional view illustrating another embodiment concerning the way of mounting wires in the imaging apparatus related to the present disclosure, according to;

FIG. 7 is a side cross-sectional view illustrating an embodiment in which an imaging optical fiber is used in the imaging apparatus related to the present disclosure;

FIG. 8 is a partially enlarged perspective view illustrating a tip portion of the imaging apparatus related to the present disclosure, the tip portion including a spherical housing that accommodates a camera cone, with wire guides being provided along an outer wall surface of a cylindrical main body;

FIG. 9 is a partially enlarged side cross-sectional view illustrating a tip portion when the imaging apparatus shown in FIG. 8 is applied to a rigid endoscope; and

FIG. 10 is a partially enlarged side cross-sectional view illustrating a tip portion when an imaging apparatus is applied to a rigid endoscope, the imaging apparatus accommodating lighting optical fibers throughout the circumference of a gap between an outer peripheral surface of a cylindrical main body and an inner peripheral surface of a shell, in place of lighting LEDs incorporated into the camera cone of the imaging apparatus illustrated in FIGS. 8 and 9.

### DETAILED DESCRIPTION OF THE PREFERRED EMBODIMENT

With reference to the drawings, hereinafter are described some embodiments of the present disclosure. However, the present disclosure shall not be limited to the embodiments described below. The components identical with or similar to each other between the drawings indicate that the components have the same configuration and thus the components are given the same reference numerals for the sake of omitting unnecessary explanation.

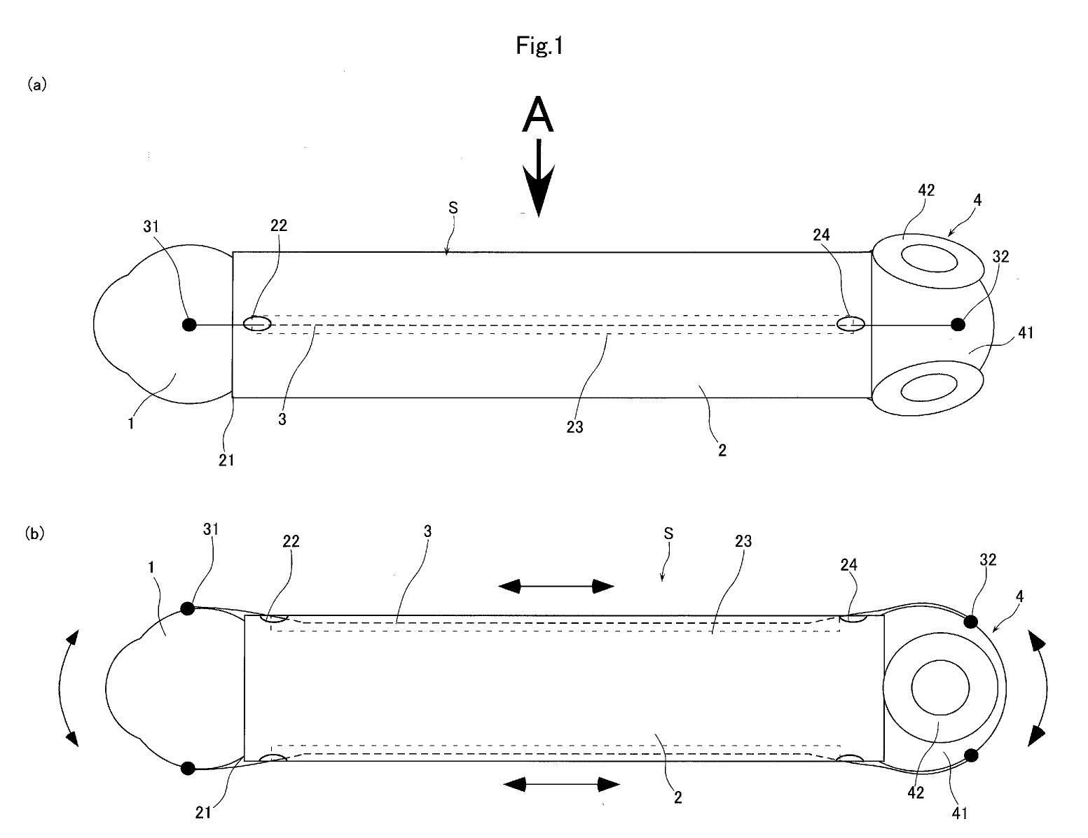

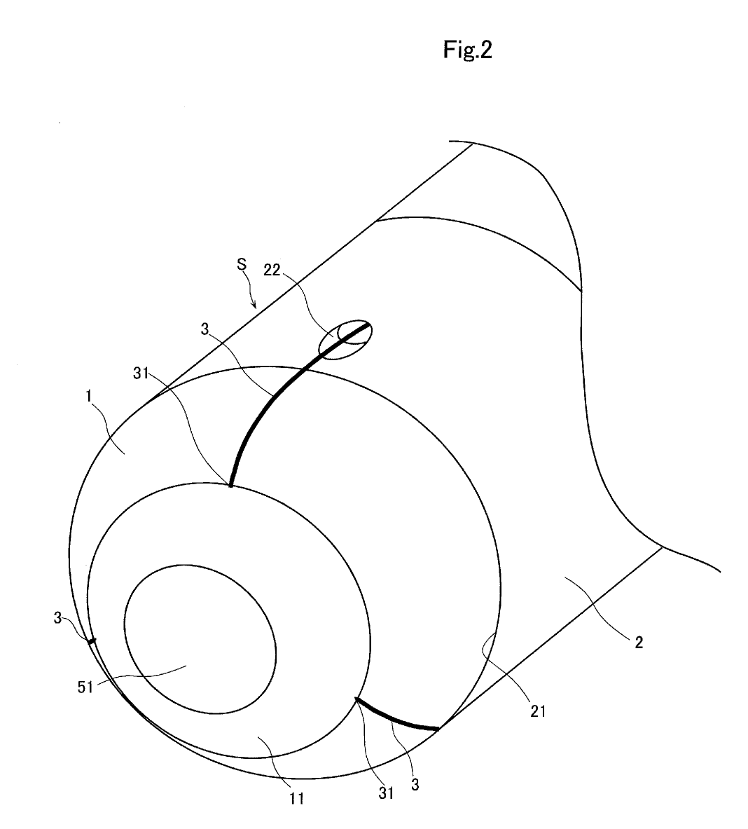

Referring to FIGS. 1 and 2, a reference numeral 1 indicates a spherical housing that holds therein an image-wise light receiving unit 5 that configures an imaging apparatus S related to the present embodiment. The image-wise light receiving unit 5 is based on a concept of including a solid-state image sensing device, such as a CCD (charge-coupled device) or a CMOS (complementary metal-oxide semiconductor), and an optical device that receives image-wise light and transmits the light to the solid-state image sensing device, as in a light-receiving surface provided in a front end portion of an optical fiber whose rear end portion is connected to the image sensing device.

The spherical housing 1 is supported by a base 21 formed at one end portion of a cylindrical main body 2 that is formed into a cylindrical shape. The base 21 shall not be limited to this mode provided that the base enables the spherical housing 1 to freely move along the surface thereof. However, as will be described later, it is preferable to make use of an end portion of the cylindrical shape in order that a component for driving the spherical housing 1 is arranged at a position distanced from the spherical housing 1. In the present embodiment, the cylindrical main body 2 is formed into a bottomless cylindrical shape, but may be formed into a bottomed cylindrical shape provided that the free movement of the spherical housing 1 is ensured. However, when a bottomed cylindrical main body 2 is used, it is required, as will be described later, to provide an opening in the bottom for passing an imaging wire therethrough, the imaging wire being connected to the spherical housing 1, so that the imaging wire will not hinder the free movement.

The cylindrical main body 2 has an outer peripheral side face which is provided with through holes 22. Each through hole 22 functions as a drive-wire-position limiter that guides a drive wire 3, whose one end is fixed to the spherical housing 1, from the outer peripheral side face near the base 21 to an inner peripheral side face. The one end of the drive wire 3 is fixed to the spherical housing 1 by a fixing portion 31.

As shown in FIG. 2, each through hole 22 passes through the cylindrical main body 2 in a throat-like manner so as to be moderately inclined in the longitudinal direction of the cylindrical main body 2, with respect to a direction perpendicular to the thickness of the side face of the cylindrical main body 2. The through holes 22 opened with this form limit the moving direction of the respective drive wires 3 only to the longitudinal direction and realize a highly accurate performance of the spherical housing 1 which is driven with the movement of the drive wires 3. The cylindrical main body 2 has the inner peripheral side face provided with wire guides 23, each of which guides the corresponding drive wire 3 passing through the corresponding through hole 22 in the longitudinal direction.

The cylindrical main body 2 has an end portion which is on the opposite side of the end portion provided with the base 21. This opposite-side end portion supports a drive section 4 which is configured by a spherical actuator. The spherical actuator is configured by a spherical rotor 41 and stators 42. The spherical actuator is configured by at least the spherical rotor 41 and the stators 42. Near the end portion of the cylindrical main body 2, the end portion supporting the spherical rotor 41, through holes 24 are formed. Each through hole 24 is formed at a position opposed to the corresponding through hole 22 in the longitudinal direction of the cylindrical main body 2 to limit the position of the corresponding drive wire 3. Each drive wire 3 that has passed through the inner peripheral side face is again exposed from the outer peripheral side face via the corresponding through hole 24 and fixed to the surface of the spherical rotor 41 by a fixing portion 32.

The spherical rotor 41 is supported by the stators 42 whose number corresponds to the number of the drive wires 3. In the present embodiment, the spherical rotor 41 is supported by three stators 42. As shown in FIG. 1 by (b) with a schematic side cross-sectional view that is a view from a direction A of (a), the movement of the spherical rotor 41 driven by the stators 42 is transmitted to the spherical housing 1 via the drive wires 3 each having an end fixed to the spherical housing 1 and the other end fixed to the spherical rotor 41 to freely drive the spherical housing 1 (see the arrows indicated in FIG. 1 by (b)).

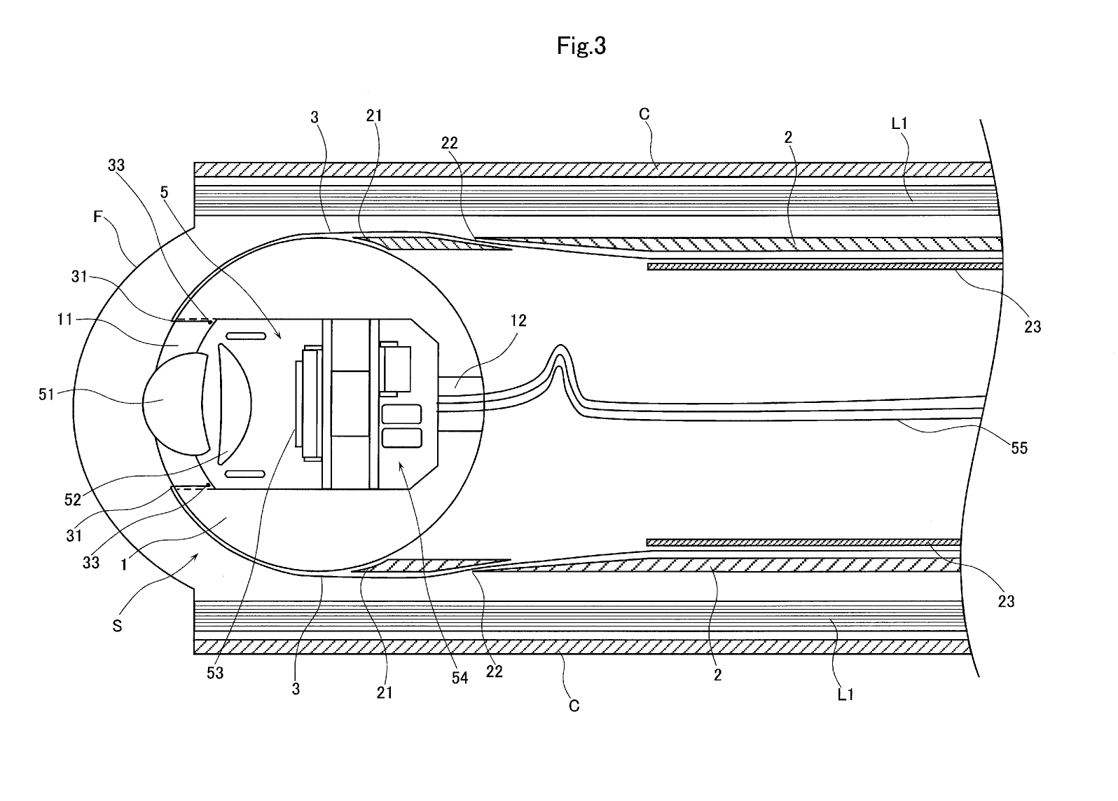

Referring to FIG. 3, the spherical housing 1 and the image-wise light receiving unit 5 are specifically described. FIG. 3 illustrates a partially enlarged tip portion in the case where the imaging apparatus S related to the present embodiment is applied to a rigid endoscope, i.e. a side cross-sectional view of the spherical housing 1. Portions common between FIGS. 1 and 2 are given the same reference numerals to omit detailed explanation.

The spherical housing 1 only has to be hollowed out to form inside an accommodation space in which the image-wise light receiving unit 5 of the imaging apparatus S can be housed, and be able to retain the shape when the spherical housing 1 is in a state of being supported by the base 21 and when it is slidably rotated, as described later, in a state of being supported by the base 21. The material of the spherical housing 1 is not particularly limited if the above requirements are met. For example, according to the purpose of use, a material may be appropriately selected, including metal, such as stainless steel or brass, an inorganic material, such as quartz glass, or an organic material, such as transparent polycarbonate resins or carbon fiber resins. In the case of a medical rigid endoscope, stainless steel may be selected because it has less sensitizing properties (metallic allergy) but has bactericidal properties for a person being examined. If the medical rigid endoscope is used in a high temperature environment, metallic cobalt may be selected.

The accommodation space inside the spherical housing 1 has an opening in a front face part that faces an imaging target. After accommodating the image-wise light receiving unit 5, a press-in front cover 11 is hermetically fitted and inserted to the opening so that the accommodation space is formed as a hermetically closed space. In the press-in front cover 11, an aspheric lens 51 configuring an optical system of the image-wise light receiving unit 5 is fitted, with a surface (convex surface) facing an imaging target being partially exposed from the outer surface of the spherical housing 1. The aspheric lens 51 has a surface (concave surface) inside the accommodation space. At a position facing this surface, a zoom lens 52 that configures the optical system is arranged. At a position facing the zoom lens 52, an imaging device 53, such as a CCD or a CMOS, is arranged to pick up an image that has been transmitted through the optical system. Behind the imaging device 53, a drive/control unit 54 of the imaging device is arranged. A bottom that defines the accommodation space, i.e. a surface opposite to the side where the optical system is arranged in the spherical housing 1, is provided with an opening 12 for drawing out an imaging wire 55. The imaging wire 55 drawn out of the opening 12 is connected to a control unit for imaging, not shown, or to a connector that establishes a wired or wireless connection with the control unit.

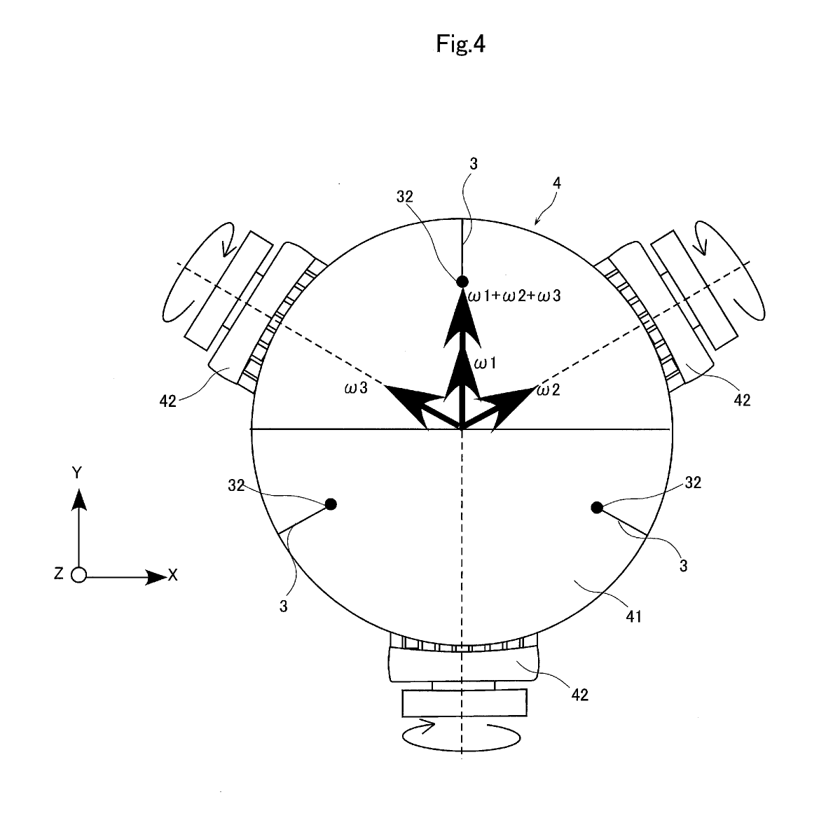

Each of the drive wires 3 has a function of transmitting the driving caused by the drive section 4 (see FIG. 4) to the spherical housing 1. The drive wires 3 only have to have this function and thus the material thereof is not limited to a specific one. However, with the operation of the spherical housing 1 by the drive section 4, the distance from each fixing portion 31 of the corresponding drive wire 3 on the spherical housing 1 side to the corresponding fixing portion 32 on the drive section 4 side (see FIG. 1) may vary. Therefore, it is preferable that the material has flexibility sufficient for absorbing the variation and has strength that can endure the flexibleness. For example, the above requirements are met by a metal wire, such as a piano wire, a wire made of a polyamide resin, such as nylon, a wire made of polyimide resin, such as kapton, or a wire made of a polyvinylidene fluoride resin. As far as the operation is not hindered, the diameter of each drive wire 3 is not required to be specified, either. For example, when a spherical housing 1 made of stainless steel with a diameter of 8 mm is used, a drive wire 3 made of a polyvinylidene fluoride resin with a diameter of 0.148 mm may be used.

Various methods may be used as a method of fixing the drive wires 3 to the spherical housing 1 as far as the methods do not hinder the operation. The fixing method shown in FIG. 3 is as follows. First, an end of each drive wire 3 to be fixed to the spherical housing 1 is melted and coagulated to form a substantially spherical engaging end portion 33 having a diameter larger than that of the drive wire 3. Grooves (not shown) for passing the respective drive wires 3 are provided in a press-in wall surface of the press-in front cover 11. Each drive wire 3 is laid along the corresponding groove, followed by inserting the engaging end portion 33 into an engaging recess provided at a rear end of the groove to thereby engage the drive wire 3 with the recess. When the press-in front cover 11 is press-fitted to the spherical housing 1 in this state, the drive wires 3 are fixed to the spherical housing 1.

Each drive wire 3 guided to the inner peripheral surface side of the cylindrical main body 2 through the corresponding through hole 22 is extended along the corresponding wire guide 23 formed in the longitudinal direction of the inner peripheral surface. The wire guide 23 suppresses the drive wire 3 from moving in a direction other than the extending direction more than necessary while the drive wire 3 is in operation. The wire guide 23 is not limited in its shape, diameter, and the like if the above function is met. For example, a groove formed in the inner peripheral surface of the cylindrical main body 2 may meet the function. Alternatively, a tubular hole formed by longitudinally boring the side face of the cylindrical main body 2 throughout its thickness so as to be continuous from the through hole 22 may provide the function. Alternatively, a tube made of a resin may be bonded to the inner wall surface to meet the function. Which of the modes should be used may be appropriately selected according to the material of the drive wires 3 and the usage of the imaging apparatus. For example, when the material of the drive wires 3 is a polyvinylidene fluoride resin, tubes made of a polyimide resin may be provided as the respective wire guides 23.

In order that the spherical housing 1 is moved (rotated) with good responsiveness and high accuracy, it is desirable that the drive wires 3 are ensured not to be sagged more than necessary when driven. On the other hand, it is also desirable the drive wires 3 have flexibility to some extent and establish a linkage between the spherical housing 1 and the drive section 4. In particular, in the case where a plurality of drive wires 3 are fixed to the spherical housing 1 and the drive section 4, the drive wires 3 are required to have the same length and be fixed with substantially even tension.

The drive wires 3 are tensed between the spherical housing 1 and the drive section 4 to not only transmit the rotation of the drive section 4 but also constantly press the spherical housing 1 against the base 21. Thus, the spherical housing 1 is constantly ensured to be in contact with the base 21 throughout the circumference of the base 21 and is prevented from coming off from the base 21 in the event that an external force is applied. Further, the drive wires 3 do not excessively press the spherical housing 1 against the base 21 and hence can freely and slidably rotate the spherical housing 1 in a state where the spherical housing 1 is supported by the base 21.

As described referring to FIGS. 1 to 3, both ends of each drive wire 3 configuring the imaging apparatus of the present disclosure are fixed being tensed between the spherical housing 1 and the drive section 4. Further, the spherical housing 1 and the drive section 4 are ensured to have a given distance therebetween being supported by the cylindrical main body 2. Therefore, the rotation of the drive section 4 can be transmitted to the spherical housing 1 with good responsiveness and high accuracy.

In addition, the shape of each through hole 22 places limitations such that the corresponding drive wire 3 is driven only in the movement in the longitudinal direction of the drive wire 3, i.e.

only in a movement in a direction of transmitting driving from the drive section 4 to the spherical housing 1. Therefore, movement with higher responsiveness and accuracy can be realized.

In the case of a rigid endoscope, the imaging apparatus S of the present disclosure is housed in a cylindrical shell C. The shell C is formed of a stainless steel tube. The shell C has an end to which a front cowl F made of transparent glass is fitted. The imaging apparatus S is housed in the shell C in such a way that the aspheric lens 51 configuring the spherical housing 1 will face the front cowl F. A gap is formed between the outer peripheral surface of the cylindrical main body 2 and the inner peripheral surface of the shell C. In the gap, lighting optical fibers L1 are accommodated being laid throughout the circumference. Desirably, the gap has a size of about 1 mm taking account of the size of the whole rigid endoscope, although it depends on the size of the lighting optical fibers L1.

Each lighting optical fiber L1 has an end face that faces an imaging target that is present in an imaging field of view, and has a rear end face to which an illumination light source (not shown), such as a LED, is connected. In this case, the lighting optical fibers L1 cast light on the imaging field of view from a position of surrounding the imaging apparatus S, and accordingly can provide illumination intensity sufficient for obtaining a good image. Further, the field of view, in which imaging is performed, is illuminated in substantially a circular shape (i.e. doughnut shape), with an illuminance at a center portion thereof being comparatively low. In general, lenses have optical characteristics which may induce a phenomenon that the imaging sensitivity around an imaging field of view is impaired. However, the configuration described above can provide an illumination distribution that compensates the low imaging sensitivity around an imaging field of view.

FIG. 4 is a top view of the drive section 4 supported by the cylindrical main body 2 as viewed in the longitudinal direction of the cylindrical main body 2. Hereinafter, the present embodiment exemplifies a configuration in which the drive section 4 is composed of the spherical rotor 41 and three stators 42 each provided with a piezoelectric device. However, this shall not impose a limitation. Accordingly, the number of the stators 42 may be appropriately determined according to the application. As will be described later, each stator 42 may desirably have an ultrasonic motor as a drive source. However, for example, two stepping motors may be perpendicularly arranged and two drive wires 3 may be connected to the rotary shaft of each of them, thereby performing two-dimensional driving, as in a gimbal structure, using four drive wires 3. Alternatively, one drive wire 3 may be connected to each of the two perpendicularly arranged stepping motors, while the drive wire 3 connected to the cylindrical main body 2 via an expansion spring may be fixed to a position symmetrical to the fixing position of the drive wire 3 connected to the stepping motor. Alternatively, the stators 42 may be manually moved without using piezoelectric devices, for the movement (rotation) of the spherical rotor 41. If the imaging apparatus is required to provide movement of three degrees of freedom as in a rigid endoscope, at least three stators 42 are necessary. In order to transmit the movement of the three stators 42 to the spherical housing 1, the drive wires 3 are required to be provided by the number corresponding to the number of the stators 42, i.e. three.

As shown in FIG. 4, the drive section is a known spherical actuator in which the spherical rotor 41 is held by the three stators 42 in a ring shape. Each stator 42 is arranged so that an axis passing through the center of the spherical rotor 41 passes through the center of the ring. Each stator 42 generates torque around its axis. An ultrasonic motor may be used as a drive source for generating the torque. The stators 42 generate torque expressed by ω1, ω2 and ω3. The operation based on the three degrees of freedom is realized by the combined torque, i.e. ω1+ω2ω3. The rotational direction and the velocity of the spherical rotor 41 are controlled by controlling the magnitude and the direction of travel of the ultrasonic vibration excited in the stators 42.

In the top view illustrated in FIG. 4, the stators 42 are arranged at regular intervals, i.e. at an interval of 120°. The drive wires 3 of the respective stators 42 are fixed at regular intervals by the fixing portions 32, at positions apart from the respective stators 42 by an angle of 60°.

The drive section 4 and the zoom lens 52 may preferably be driven by ultrasonic motors that do not use a magnetic material so that the motors can operate under the use of MRI (magnetic resonance imaging system).

The drive section 4 may have a configuration in which, an operating stick, not shown, is mounted to the spherical rotor 41 in place of the stators 42 to enable the surgeon to directly rotate the spherical rotor 41 by hand. With this configuration, a movement to an observation field of view intended by the surgeon can be completed without a time lag and can be quickly locked at the intended observation field of view. Accordingly, fatigue of the surgeon is reduced in carrying out long surgical operations.

As the fixing position of each drive wire 3 on the spherical housing 1 is made closer to a vertex of the spherical housing 1, the movable range of the spherical housing 2 can be more expanded. In this case, the vertex is defined to be the center of the outer surface of the spherical housing 1 as viewed from the longitudinal direction of the cylindrical main body 2. However, when the fixing positions are close to the vertex, the fixing portions 31 of the drive wires 3 may be included in the view angle in imaging and thus the fixing portions 31 may be shown in the picked up image. When the movable range is expanded as mentioned above, the drive wires 3 fixed on the opposite side of the rotational direction of the spherical housing 1 will have a large length of external exposure along the surface of the spherical housing 1. The drive wires 3 externally exposed along the surface of the spherical housing 1 are likely to move in a direction other than the longitudinal direction as well. Moreover, the fixing portions 31 of the externally exposed drive wires 3 are distanced from the base 21 and hence the force for pressing the spherical housing 1 against the base 21 is impaired. As a result, the stability of the spherical housing 1 against an external force becomes low with such a rotational position.

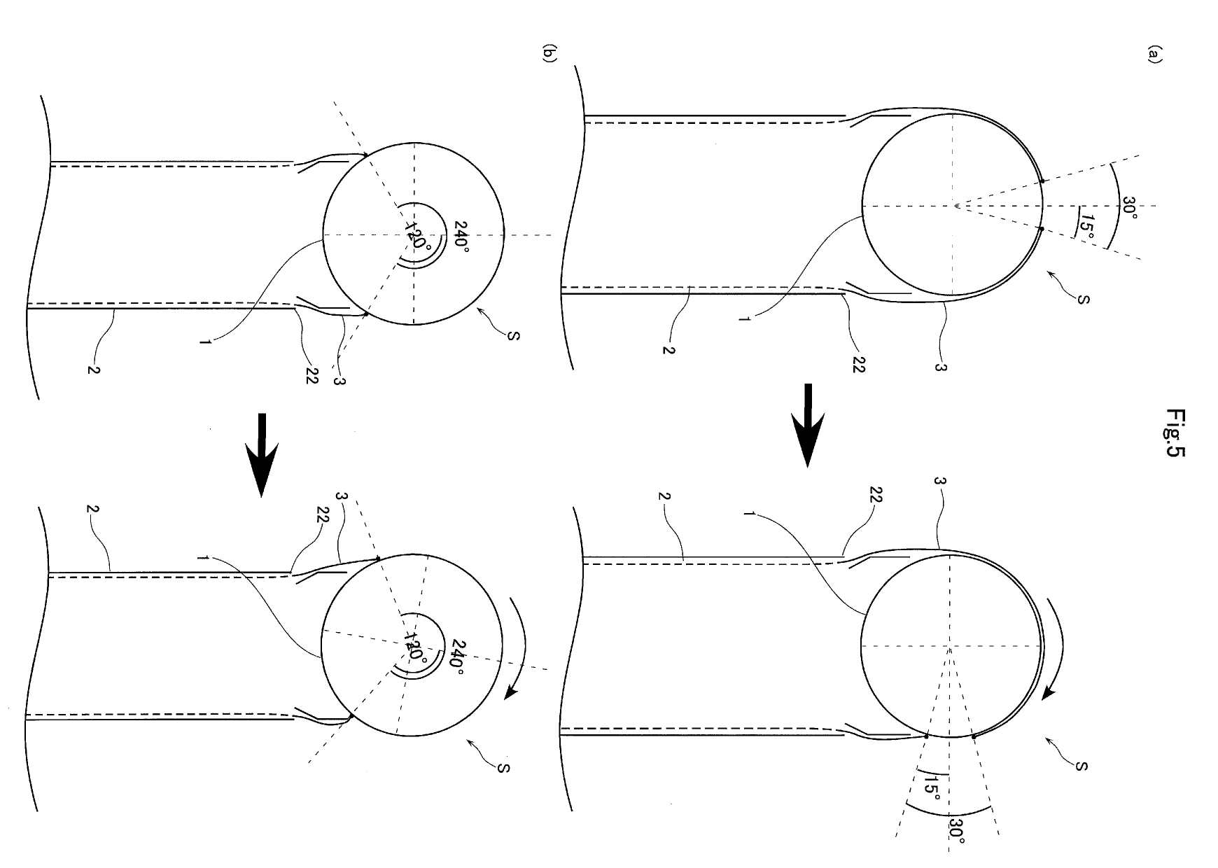

On the other hand, in order to avoid the above disadvantages, the fixing positions of the drive wires 3 may be brought to positions apart from the vertex. In this case, however, the movable range is narrowed and accordingly a good observation field of view cannot be obtained. In this regard, as shown in FIG. 5, each drive wire 3 may preferably be mounted at a position apart from the vertex by an angle of 30°. Specifically, this is a position that forms a narrow angle of 30° or more between two sides that connect the center of the spherical housing 1 to the fixing parts of the mutually adjacent drive wires 3 on the surface of the spherical housing 1 (see (a) of FIG. 5), a position apart from the vertex by an angle of 120°. More specifically, the position falls in a rage of the narrow angle of 240° or less (see (b) of FIG. 5).

The diameter of an end of the rigid endoscope may be changed depending on the purpose of usage or the size such as of a device incorporated in the spherical housing 1. However, in general, the diameter of an end of the rigid endoscope approximately ranges from 5 mm to 10 mm and accordingly the diameter of the spherical housing 1 may also approximately range from 5 mm to 10 mm. On the other hand, the drive section 4, which is disposed far from the imaging section, has no constraint on the size. Thus, the size of the drive section 4 may be selected so as to be suitable for an actuator that enables stable operation. For example, in the present embodiment, the diameter of the spherical rotor 41 is about 8 mm.

According to the present embodiment, the spherical housing 1 is freely moved via the drive wires 3 and hence the drive section 4 is no longer required to be provided at the spherical housing itself or near the spherical housing. Thus, the size of the imaging section inserted into a narrow observation area is reduced without being constrained by the size of the mechanism for driving the imaging section.

In the present embodiment, each through hole 22 is provided in the base 21 to serve as a drive-wire-position limiter that limits the movement of the corresponding drive wire 3 only in the longitudinal direction, so that, when the spherical housing 1 is moved by the drive section 4, the drive wire 3 is suppressed from being moved in a direction other than the longitudinal direction. Thus, the spherical housing 1 is made freely movable along its surface via the drive wires 3 whose movement is limited only in the longitudinal direction. Accordingly, a field of view can be moved with good accuracy in a wide range even in a narrow space.

In the present embodiment, the base 21 is formed in a cylindrical shape, with one end portion of the cylindrical shape supporting the spherical housing 1. Further, each through hole 22 is formed to serve as a drive-wire-position limiter to thereby guide the corresponding drive wire 3 fixed to the spherical housing 1, from the outer peripheral side face of the cylindrical shape to the inner peripheral side face thereof. Further, an end of the drive wire 3 passing through the inner peripheral side face is fixed to the drive section 4 that is supported by an end portion of the cylindrical shape, the end portion being on the opposite side of the end portion supporting the spherical housing 1. Thus, the drive section 4 can be disposed far from the spherical housing 1. Accordingly, the size of the imaging section inserted into a narrow observation area is reduced without being constrained by the size of the drive mechanism of the drive section 4.

In the present embodiment, the drive section 4 is a spherical actuator, with an end of each drive wire 3 being fixed to the surface of the spherical actuator. Thus, the movement of the spherical housing 1 can be in synchrony with the movement of the spherical actuator without being intervened by a complicated drive conversion mechanism. Accordingly, the size of the imaging apparatus as a whole including the drive section 4 is reduced.

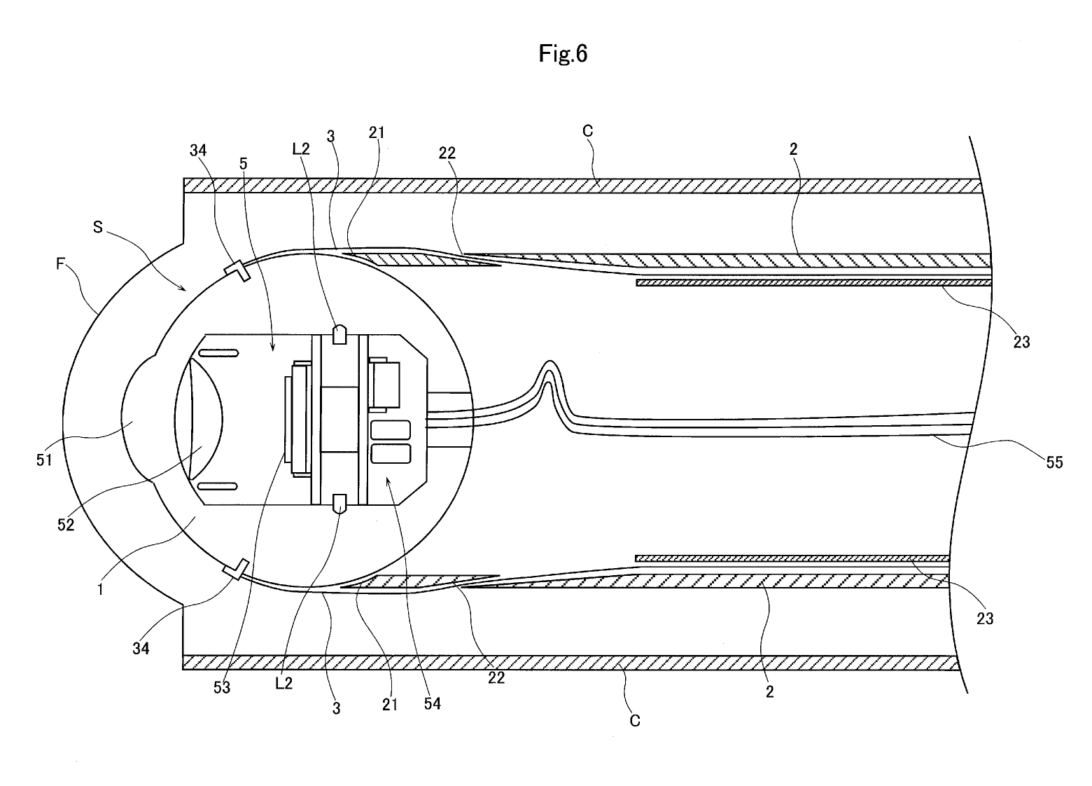

Referring to FIG. 6, another embodiment of the present disclosure is described. FIG. 6 illustrates an enlarged tip portion in the case where the imaging apparatus S related to another embodiment of the present disclosure is applied to a rigid endoscope, i.e. illustrates a side cross-sectional view of the spherical housing 1. The components common between FIGS. 1 to 3 are given the same reference numerals to omit detailed explanation.

In FIG. 6, the spherical housing 1 is configured by a translucent material, in place of the lighting optical fibers L1 used in FIG. 3, with lighting LEDs L2 being provided inside the spherical housing 1. In this case, the light of the lighting LEDs L2 is internally guided through the spherical housing 1 to illuminate an imaging field of view.

With this configuration, the movement of the field of view, in which imaging is performed by the image-wise light receiving unit 5 that is fixed to the spherical housing 1, and the movement of an illumination field of the lighting LEDs L2 are in synchrony with the movement of the spherical housing 1. Accordingly, the field of view, in which imaging is enabled, is broadened and thus good illumination intensity is provided over the entire field of view when the movable range of the spherical housing 1 is expanded.

The drive wires 3 are fixed to the spherical housing 1 using a method in which an end of each of the drive wires 3 is fixed onto the surface of the spherical housing 1 using a fixing member 34, such as a screw. In this way, fixation of the drive wires 3 onto the spherical housing 1 is facilitated, thereby enhancing productivity.

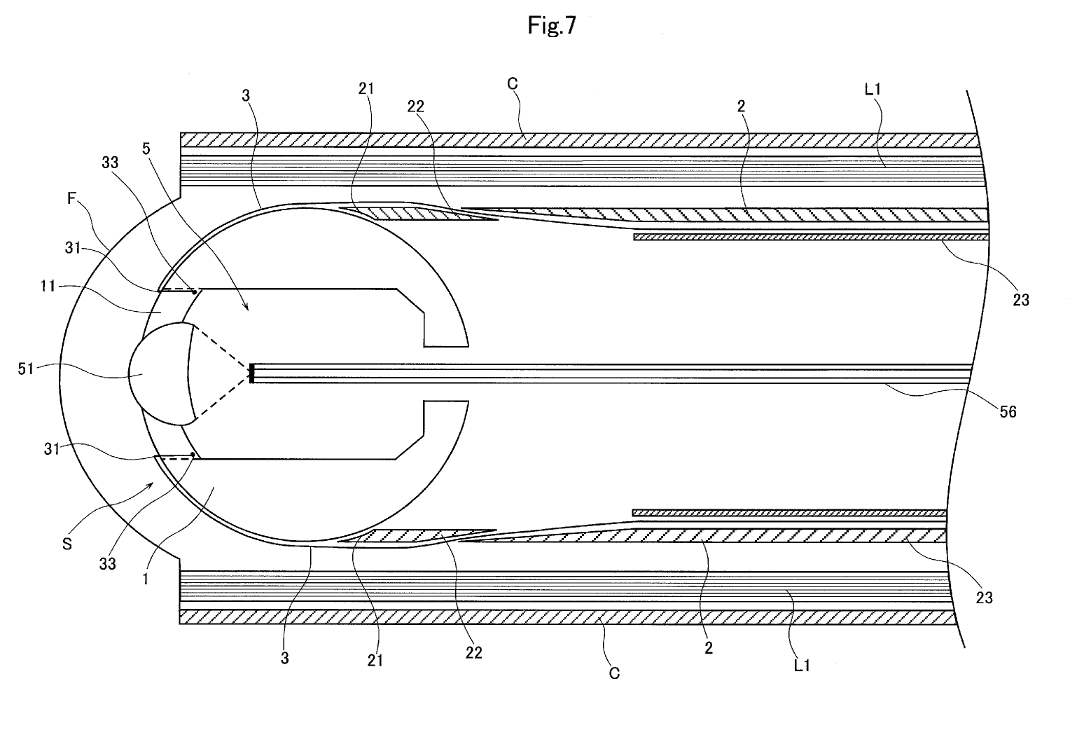

Referring to FIG. 7, another embodiment of the present disclosure is described. FIG. 7 illustrates an enlarged tip portion in the case where the imaging apparatus S related to another embodiment of the present disclosure is applied to a rigid endoscope, i.e. illustrates a side cross-sectional view of the spherical housing 1. The components common between FIGS. 1 to 3 and FIG. 6 are given the same reference numerals to omit detailed explanation.

In the embodiments shown in FIGS. 3 and 6, the image sensing device 53 and the drive/control unit 54 of the image sensing device are incorporated in the accommodation space of the spherical housing 1. However, in FIG. 7, the image sensing device 53 and the drive/control unit 54 of the image sensing device are ensured not to be provided in the image-wise light receiving unit 5 in the accommodation space, in order that normal operation of the image sensing device 53 and the drive/control unit 54 of the image sensing device will not be hindered being affected by the ultimate environment such as of high temperature, high pressure or high radiation.

The accommodation space of the spherical housing 1 only accommodates an imaging optical fiber 56 which is arranged such that its light-receiving surface will be located at the focal position of the aspheric lens 51. The imaging optical fiber 56 has an end opposite to the light-receiving surface, which end is provided with an image sensing device, not shown. With this configuration, when the apparatus is used under a considerably high temperature environment, quartz glass may be used as an optical material for the aspheric lens 51 and the optical fiber, metal may be used as a mechanical material for the spherical housing 1 and the base, and metal wires may be used for the drive wires 3.

Further, a three-band type optical fiber (that transmits received light by splitting the light into R, G and B light beams) may be used as the imaging optical fiber 56. Through this imaging optical fiber 56, R, G and B light beams may be imaged by respective three image sensing devices and combined by an image processor, not shown, to thereby obtain a picked up image with good color reproducibility.

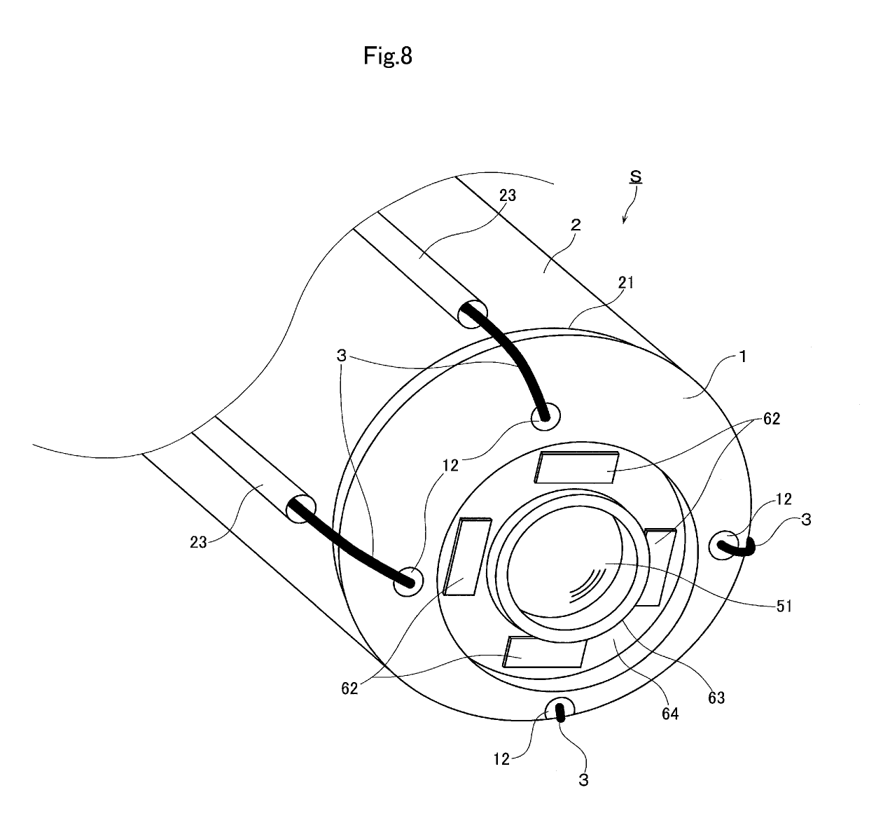

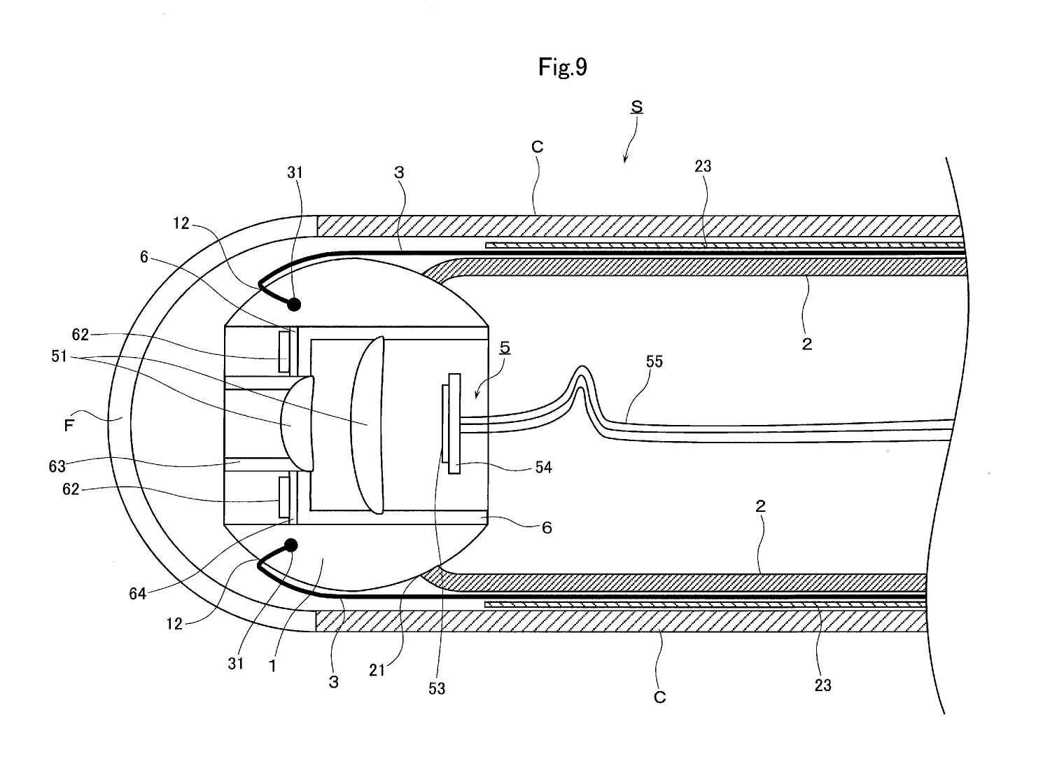

Referring to FIGS. 8 and 9, another embodiment of the present disclosure is described. FIG. 8 is a partially enlarged perspective view illustrating a tip portion of the imaging apparatus S related to the embodiment of the present disclosure. FIG. 9 is a partially enlarged side cross-sectional view illustrating a tip portion when the imaging apparatus S related to the embodiment of the present disclosure is applied to a rigid endoscope. The components common between the embodiments shown in FIGS. 1 to 7 are given the same reference numerals to omit detailed explanation.

In the embodiment shown in FIGS. 2 and 3, the image-wise light receiving unit 5 of the imaging apparatus S is directly accommodated inside the accommodation space hollowed out in the spherical housing 1. On the other hand, the embodiment shown in FIGS. 8 and 9 is different from the embodiment shown in FIGS. 2 and 3 in that a camera cone 6, which houses in advance the image-wise light receiving unit 5 and lighting LEDs 62 as self-luminous type lighting devices, is inserted, for fixation, into the accommodation space hollowed out in the spherical housing 1, and the wire guides 23 are provided along the outer wall surface of the cylindrical main body 2 while the respective drive wires 3 are also extended along the outer wall surface of the cylindrical main body 2.

The camera cone 6 has a cylindrical body made of metal or a resin, with its outer diameter being slightly smaller than the diameter of the accommodation space hollowed out in the spherical housing 1. The camera cone 6 has an interior for arranging the aspheric lens 51, the image sensing device 53 and the drive/control unit 54 of the image sensing device, which configure the image-wise light receiving unit 5, and the lighting LEDs 62.

The camera cone 6 has a front opening, i.e. an opening on the side of an object as an imaging target, provided with the aspheric lens 51 which is surrounded by the plurality of lighting LEDs 62 supported by a cored circular LED base 64. A lens hood 63 is provided to shield the aspheric lens 51 from the lighting LEDs 62. The lens hood 63 surrounds the aspheric lens 51 and is projected forward with reference to the position at which the aspheric lens 51 in the camera cone 6 is set up. Thus, the lens hood 63 shields the entry of the illumination light of the lighting LEDs 62 into the aspheric lens 51 to prevent the occurrence of lens flare. In the present embodiment, the lens hood 63 is integrally formed with the camera cone 6.

The camera cone 6 has a rear opening, i.e. an opening on the opposite side of the object as an imaging target, through which imaging wires 55 are drawn out of the drive/control unit 54 of the image sensing device, the unit 54 supporting the image sensing device 53. The imaging wires 55 include a power supply wire for supplying power to the lighting LEDs 62, the power supply wire being connected to a wire, not shown, in the LED base 64.

In assembling the imaging apparatus related to the present embodiment, the aspheric lens 51, the image sensing device 53 and the drive/control unit 54 of the image sensing device, which configure the image-wise light receiving unit 5, and the lighting-LEDs 62 are firstly mounted to the camera cone 6, for the adjustment of the positional relationship therebetween. Then, the camera cone 6 is inserted into the spherical housing 1 and adjusted to a position at which imaging is well performed, and then fixed. This assembling procedure enables highly accurate and efficient assemblage.

In the present embodiment, a titanium alloy is used for the drive wires 3. The titanium alloy having high durability and heat resistance may preferably have shape-memory effect. Exerting the shape-memory effect when the titanium alloy is subject to high temperature sterilization process, strain of the drive wires 3 caused by long time use can be easily corrected.

When the drive wires 3 are fixed to the spherical housing 1 in the present embodiment, tin that is in substantially spherical shape is fused to an end portion of each drive wire 3, the end portion being fixed to the spherical housing 1, to form the engaging end portion 31. Then, the drive wire 3 is passed through an engaging hole 12 punched in the spherical housing 1 so that the engaging end portion 31 is positioned on the accommodation space side of the spherical housing 1. In this case, since the diameter of the engaging end portion 31 is made larger than the diameter of the engaging hole 12, the engaging end portion 31 engages with the engaging hole 12 to allow the drive wire 3 to be tense between the spherical housing 1 and the drive section 4.

The wire guides 23 provided along the outer wall surface of the cylindrical main body 2 in the present embodiment are each formed by fitting a tube made of a metal mesh to a tube made of a polyimide resin. Such wire guides 23 are favorable in that they have high heat resistance and reduce frictional resistance caused between each wire guide 23 and the corresponding drive wire 3. The wire guides 23 are each fixed to the outer wall surface of the cylindrical main body 2 such as by performing bonding or by performing fastening using a fastener, not shown. In the present embodiment, each of the drive wires 3 is inserted into the corresponding wire guide 23 provided at the outer wall surface of the cylindrical main body 2. This reduces portions in the drive wires 3, which suffer from flexure or friction. Thus, the drive wires 3 and the wire guides 23 will have high durability, while the drive wires 2 are permitted to have small drive resistance to enhance operability.

In the present embodiment, each wire guide 23 has a tip opening positioned almost reaching the base 21 that is in contact with the spherical housing 1. Thus, the positional shifting of the drive wire 3 is limited in the circumferential direction of the cylindrical main body 2. In other words, the tip opening of each wire guide 23 functions as a drive-wire-position limiter.

The imaging apparatus S related to the present embodiment is housed in the shell C, which is similar to the rigid endoscope shown in FIG. 3, but is enabled, in the present embodiment, to allow insertion and removal of the imaging apparatus S from the shell. The shell C has an inner peripheral surface which is provided with a guide member, not shown, for engagement with an engaging member, not shown, provided to the imaging apparatus S. The imaging apparatus S is inserted into or removed from the shell C in a state of being engaged with the guide member of the shell C. Thus, positional relation with the shell C or the gap is ensured to be favorable for imaging. With this configuration, the imaging apparatus S can be changed in a state where the shell C is kept being placed in the body cavity of a person being examined. Being less invasive, this configuration enables use of a plurality of imaging apparatuses having the respective functions (e.g. angular field or magnification) according to the purposes of imaging, repeating insertion and removal of the imaging apparatuses in a state where the shell C is kept being steadily placed at the same site in the body cavity. In this way, imaging is efficiently performed in the range of a single field of view.

According to the present embodiment, the image-wise light receiving unit 5 and the lighting LEDs 62 are housed in advance in the camera cone 6 and in this state the camera cone 6 is inserted into the accommodation space of the spherical housing 1 and fixed. Thus, mounting and adjustment of the image-wise light receiving unit 5 and the lighting LEDs 62 are facilitated and highly accurately performed. Further, according to the present embodiment, the plurality of lighting LEDs 62 are adjacently set up around the image-wise light receiving unit 5. Accordingly, illuminance and its distribution can be favorably retained in all the observation fields of view in which the spherical housing 1 is rotatable. Thus, imaging which is excellent in resolution and color reproducibility can be easily performed.

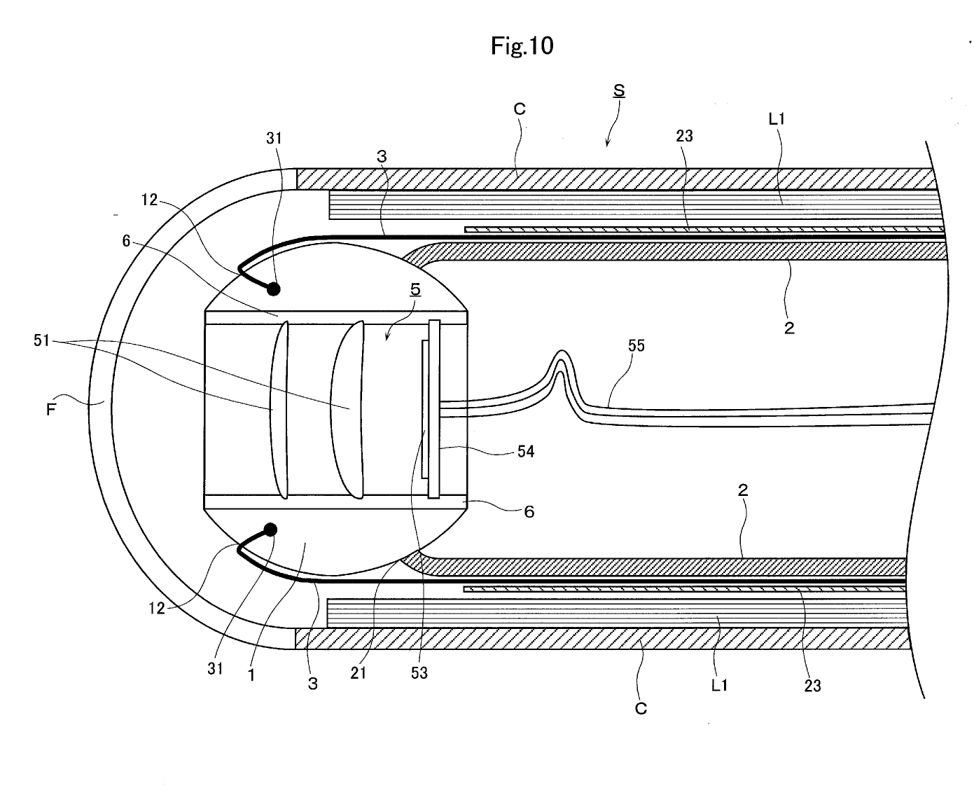

Referring to FIG. 10, another embodiment related to the present disclosure is described. FIG. 10 is a partially enlarged side cross-sectional view illustrating a tip portion when the imaging apparatus S related to the embodiment of the present disclosure is applied to a rigid endoscope. The components common between the embodiments shown in FIGS. 1 to 9 are given the same reference numerals to omit detailed explanation.

In the present embodiment, similar to the embodiment shown in FIGS. 8 and 9, the camera cone 6 incorporated in advance with the image-wise light receiving unit 5 is inserted, for fixation, into the accommodation space that is cylindrically hollowed out in the spherical housing 1, and the wire guides 23 are provided along the outer wall surface of the cylindrical main body 2, while the respective drive wires 3 are also extended along the outer wall surface of the cylindrical main body 2. Further, the imaging apparatus S is housed in the cylindrical shell C such that the apparatus can be inserted into or removed from the shell.

The present embodiment is different from the embodiment shown in FIGS. 8 and 9 in that the lighting optical fibers L1 that function as lighting power sources are provided instead of the lighting LEDs 62 that are incorporated in the camera cone 6. The lighting optical fibers L1 are accommodated throughout the circumference of the gap between the outer peripheral surface of the cylindrical main body 2 and the inner peripheral surface of the shell C. The lighting optical fibers L1 transmit emitted light of a light source, not shown, to cast illumination light towards a person being examined that is an imaging target.

According to the present embodiment, the light source is provided at a rearward place in the imaging apparatus, i.e. provided in a place far from a person being examined that is an imaging target. The emitted light of the light source is transmitted by the light optical fibers L1 so that illumination light is cast on a person being examined that is an imaging target. This minimizes the change in color temperature of the illumination light due to the generated heat of the light source, or minimizes the change in imaging characteristics of the image sensing device 53 due to the temperature rise in the imaging apparatus S, when imaging is performed over a long period of time. Thus, imaging which is excellent in resolution and color reproducibility can be easily performed over a long period of time.

In the embodiments shown in FIGS. 2 to 10, the imaging apparatus related to the present disclosure is applied to a rigid endoscope. However, the imaging apparatus related to the present disclosure may also be applied to a flexible endoscope. In general, a flexible endoscope includes an operating section and an insertion section continuing into the operating section. The insertion section includes, from the operating section side, a flexible tube portion formed of a flexible cylindrical body, a bendable portion that can be bent in a given direction by an operating wire bridged in relation to the operating section, and a tip portion that holds an image sensing device or an observation optical system. The spherical housing of the imaging apparatus related to the present disclosure is mounted to the tip portion of the flexible endoscope and the drive section is driven being interlocked with the operation of the operating section of the flexible endoscope.

As described above, the image-wise light receiving unit 5 is housed inside the spherical housing 1 and the spherical housing 1 is freely moved to enable movement, such as panning or tilting, in an imaging field of view without rotating or moving the imaging apparatus as a whole.

### DESCRIPTION OF SYMBOLS

1 Spherical housing
2 Cylindrical main body
3 Drive wire
4 Drive section
5 Image-wise light receiving unit
21 Base
22 Through hole

What is claimed is:

1. An imaging apparatus wherein the apparatus comprises: an image-wise light receiving means; a spherical housing that holds therein the image-wise light receiving means; a base that supports the spherical housing and enables the spherical housing to freely move along a surface thereof; a drive wire having an end fixed to the spherical housing; and a drive section to which the other end of the drive wire is fixed to drive the free movement of the spherical housing via the drive wire.

2. The imaging apparatus according to claim 1, wherein the base has a drive-wire-position limiter that limits the movement of the drive wire only in a longitudinal direction.

3. The imaging apparatus according to claim 2, wherein the base is formed in a cylindrical shape, with one end portion of the cylindrical shape supporting the spherical housing; the drive-wire-position limiter is formed of a through hole that guides the drive wire fixed to the spherical housing, from an outer peripheral side face of the cylindrical shape to an inner peripheral side face thereof; the drive wire passing through the inner peripheral side face has an end fixed to the drive section that is supported by an end portion of the cylindrical shape, the end portion being on an opposite side of the end portion supporting the spherical housing.

4. The imaging apparatus according to claim 2, wherein the base is formed in a cylindrical shape, with one end portion of the cylindrical shape supporting the spherical housing; and the drive-wire-position limiter is provided in the outer peripheral side face of the cylindrical shape.

5. The imaging apparatus according to claim 1, wherein the drive section is a spherical actuator and an end of the wire is fixed to a surface of the spherical actuator.

6. The imaging apparatus according to claim 1, wherein the drive wire is tensed between the spherical housing and the drive section so as to retain contact between the base and the spherical housing.

7. The imaging apparatus according to claim 1, wherein the imaging apparatus has a camera cone that holds therein the image-wise light receiving means and is held inside the spherical housing.

8. The imaging apparatus according to claim 1, wherein the imaging apparatus has a lighting device that illuminates an imaging target; and the spherical housing holds the lighting device.

9. A rigid endoscope which comprises an imaging apparatus which comprises: an image-wise light receiving means; a spherical housing that holds therein the image-wise light receiving means; a base that supports the spherical housing and enables the spherical housing to freely move along a surface thereof; a drive wire having an end fixed to the spherical housing; and a drive section to which the other end of the drive wire is fixed to drive the free movement of the spherical housing via the drive wire.

10. The rigid endoscope according to claim 9, wherein the base has a drive-wire-position limiter that limits the movement of the drive wire only in a longitudinal direction.

11. The rigid endoscope according to claim 10, wherein the base is formed in a cylindrical shape, with one end portion of the cylindrical shape supporting the spherical housing; the drive-wire-position limiter is formed of a through hole that guides the drive wire fixed to the spherical housing, from an outer peripheral side face of the cylindrical shape to an inner peripheral side face thereof; the drive wire passing through the inner peripheral side face has an end fixed to the drive section that is supported by an end portion of the cylindrical shape, the end portion being on an opposite side of the end portion supporting the spherical housing.

12. The rigid endoscope according to claim 10, wherein the base is formed in a cylindrical shape, with one end portion of the cylindrical shape supporting the spherical housing; and the drive-wire-position limiter is provided in the outer peripheral side face of the cylindrical shape.

13. The rigid endoscope according to claim 9, wherein the drive section is a spherical actuator and an end of the wire is fixed to a surface of the spherical actuator.

14. The rigid endoscope according to claim 9, wherein the drive wire is tensed between the spherical housing and the drive section so as to retain contact between the base and the spherical housing.

15. The rigid endoscope according to claim 9, wherein the rigid endoscope has a camera cone that holds therein the image-wise light receiving means and is held inside the spherical housing.

16. The rigid endoscope according to claim 9, wherein the rigid endoscope has a lighting device that illuminates an imaging target; and the spherical housing holds the lighting device.
